(19) United States
(12) Patent Application Publication
(10) Pub. No.: US 2007/0105160 A1
(43) Pub. Date: May 10, 2007

(54) Detection of intracellular enzyme complex

(75) Inventors: Peter A. Fung, Sunnyvale, CA (US); Philip A. Kobel, Campbell, CA (US); Richard Eglen, Los Altos, CA (US)

Correspondence Address: PETERS VERNY , L.L.P., 425 SHERMAN AVENUE, SUITE 230, PALO ALTO, CA, 94306, US

(73) Assignee: DiscoveRx

(21) Appl. No.: 11/585,498

(22) Filed: Oct. 24, 2006

Related U.S. Application Data
(60) Provisional application No. 60/730,089, filed on Oct. 24, 2005.

(51) Int. Cl. G01N 33/567; C12Q 1/34

(52) U.S. Cl. 435/7.2; 435/18

(57) ABSTRACT

Methods and compositions are provided for determining intracellular translocation of proteins employing β-galactosidase fragments that independently complex to form an active enzyme. Cells are used having two fusion constructs: one fragment bound to a protein of interest; and the other fragment bound to a compartment localizing signal. The cells are used to screen compounds for their effect on translocation, where a substrate containing high ionic strength solution is used for detection of the enzyme complex.

### CROSS-REFERENCE TO RELATED APPLICATIONS

This application claims priority from U.S. Provisional Patent Application No. 60/730,089 filed on Oct. 24, 2005, which is hereby incorporated by reference in its entirety.

### STATEMENT OF GOVERNMENTAL SUPPORT

None.

### REFERENCE TO SEQUENCE LISTING

Applicants assert that the paper copy of the Sequence Listing is identical to the Sequence Listing in computer readable form found on the accompanying computer disk. Applicants incorporate the contents of the sequence listing by reference in its entirety.

### BACKGROUND OF THE INVENTION

1. Field of the Invention

The field of this invention is high throughput screening assays for intracellular events.

2. Related Art

The determination of intracellular events has become of increasing interest for research in the determination of cellular pathways and events and for screening compounds for their biological activity. Numerous techniques have been described in the literature for detecting intracellular events. One event that has assumed greater importance in understanding cellular pathways and in identifying potentially therapeutic compounds is translocation. In translocation, when a cell is stimulated by an external event, one or more cellular components will move from one cellular compartment or site to another compartment or site. As a result of this translocation, pathways may be induced or inhibited, transcription may be initiated or inhibited, cellular components may be degraded or modified, or other events may occur, as well as combinations thereof.

In screening for potential therapeutic agents, one is interested in high throughput screening, using multiwell plates. These plates can be handled robotically, the wells filled simultaneously, the plates stacked and moved in large numbers and the readings of each plate performed simultaneously, substantially guaranteeing that the various stages are performed under the same conditions. In this way while plates are incubating, other plates may be manipulated, read, filled or otherwise managed, so that the equipment is efficiently used. Assay protocols and reagents must be robust, reliable and provide robust signals at low concentrations of the reagents and candidate compounds.

To a substantial degree, translocation analyses have relied upon the use of fluorescent protein tags. This approach has many shortcomings in that the fluorescent proteins are large and may interfere with the events of interest, the fluorescence intensity is low, so that relatively large amounts of the label are required for detection and detection requires observing the signal at a site in the cell, which is difficult, subject to background interference and can be misinterpreted. Therefore, there remains a need for versatile, accurate and automatable methods for performing high throughput screening.

3. Relevant Literature

Descriptions of the use of β-galactosidase fragments is extensively found in the patent and scientific literature, see, for example, WO 00/039348, as indicated above, describes a protease assay where the marker is a β-galactosidase fragment fused to a protein having a specific protease cleavage site. There are numerous other references concerned with the use of β-galactosidase fragments in assay systems. The following are illustrative. Douglas, et al., Proc. Natl. Acad. Sci. USA 1984, 81:3983-7 describes the fusion protein of ATP-2 and lacZ; and WO92/03559 describes a fusion protein employing α-complementation of β-galactosidase for measuring proteinases. Assays based on complementation of enzyme fragments fused to interacting proteins that, when the interacting proteins are complexed, provide an active enzyme, may be found in a review by Rossi, et al., 2000 Trends Cell Biol. 10, 119-22. Fung et al., “A Homogeneous Cell-Based Assay to Measure Nuclear Translocation Using β-Galactosidase Enzyme Fragment Complementation,” ASSAY and Drug Dev. Tech. 4(3):263-272 (2006) further describes certain reagents and procedures relating to enzyme fragment complementation.

### BRIEF SUMMARY OF THE INVENTION

Intracellular assays are performed by using β-galactosidase fragments that independently complex to form an active enzyme, with one fragment fused to a protein of interest. The cells to be assayed express the fused fragment and the non-fused fragment with one of the fragments being located in a predefined location, e.g., compartment or site, while the other fragment is at a different location. When stimulated, the fragment at the different location moves to the predefined location to form an active enzyme complex. Permeabilizing high ionic strength buffer and a substrate resulting in a luminescent product is added in large volume compared to the cell-containing solution and the luminescence of the solution read.

### BRIEF DESCRIPTION OF THE DRAWINGS

FIG. 1 is a response curve to glucocorticoid agonists and inhibitors with cells modified with expression constructs for ED fused to glucocorticoid receptor (GR) and EA fused to a nuclear location signal (NLS);

FIG. 2 is a graph of the cells described in FIG. 1 in response to geldenmycin after activation with dexamethasone; and

FIGS. 3A and 3B are graphs of the response of the cells to the GR agonist dexamethasone comparing a chemiluminescent product producing substrate and a fluorescent product producing substrate, respectively.

### DETAILED DESCRIPTION OF THE PREFERRED EMBODIMENT

Methods and reagents are provided for performing intracellular assays to detect the status of cells, particularly as to translocation events. The primary components of the assay are genetically modified cells and a reagent composition for obtaining a signal in relation to a particular cellular event. The genetically modified cells have two expression constructs: a fusion protein of one fragment of β-galactosidase fused to a protein of interest; an expression construct of the other fragment of β-galactosidase, fused to a location signal sequence, unless the compartment is the cytosol. The reagent composition is a high ionic strength aqueous solution comprising a plurality of inorganic salts, an organic buffer, a magnesium salt for the enzyme and a β-galactosidase substrate providing a luminescent product.

The β-galactosidase fragments are referred to as α or ED and as ω or EA. The ED is generally the smaller fragment and will usually be from about 35 to 90, more usually from about 40 to 60, amino acids, including at least about amino acids 5 to 40 of the N-terminal portion of the enzyme, while the EA will generally lack a major portion of the N-proximal terminus, but include the remainder of the enzyme. The EA in the absence of ED will be relatively inactive, providing less than about 5% of the activity of the EA complexed with the ED. For further description of the β-galactosidase enzyme fragments and their use in assays, see, for example, U.S. Pat. Nos. 4,378,428; 4,708,929; 5,037,735; 5,106,950; 5,362,625; 5,464,747; 5,604,091; 5,643,734; and PCT application nos. WO96/19732; and WO98/06648.

The ED and EA will independently complex to form an active enzyme without the aid of other binding proteins to which they are fused. Thus, in the cell in the absence of additional binding affinity the fragments form an active enzyme. This is in contrast to what has been referred to as a weak affinity, where in the absence of binding of two polypeptides fused to different fragments, the ED and EA will not form a significant amount of active enzyme.

Generally, the small fragment will be fused to the target protein. The ED may be fused at any convenient site of the target protein, where it does not interfere with the function of the target protein, including the translocation, and is available for complexing with EA.

Proteins of interest are proteins in the cell that upon a stimulus translocate from one compartment to another. Among surface membrane proteins of interest are the glucocorticoid receptor, glucose transporter, steroid receptors, etc. The site to which translocation occurs may be the nucleus, Golgi apparatus, cytoplasmic membrane, or other cellular compartment.

The EA may be modified by fusing one or more control signal sequences to the EA. For example, when it is wished that the EA be localized to the nucleus, an NLS would be fused to the EA, and optionally an NRS. The NRS is a nuclear retention signal, as described e.g. in Mol Cell Biol. 2002 October; 22(19): 6871-6882. For other locations, other control signal sequences may be employed, as appropriate.

The primary function of the subject invention is the detection of compounds that stimulate or inhibit stimulation of translocation. Therefore, the compounds will bind to a target, usually the receptor-binding site, although allosteric effects may also be evaluated. Alternatively, the compound can inhibit a known binding compound, such as the natural ligand, to measure its inhibition. In this way, candidate compounds may be found to have desirable biological activity, which compounds may be further investigated as drugs for a variety of indications.

The method comprises growing the cells in an appropriate medium comprising the environment to the desired number of cells in a small volume, followed by providing the desired stimulus, e.g. candidate compound, to provide the assay sample. The volume will generally not exceed about 250 μl, usually not more than about 200 μl, and generally at least about 25 μl, where volume of the reagent addition will generally dilute the cell medium less than about 1:1, usually less than about 0.5:1. When the reagent is dry, there will be no dilution. After incubating the assay sample for sufficient time for the event of interest to occur, generally from about 1 h to about 1 day, a reagent solution is added to the assay sample and one or more readings taken of the product from the substrate. The ratio of dilution will be not more than about 1:2, usually in the ration of about 1:0.25 to 1:2, more usually 1:1 and as little at 1:0.25 or less. This dilution factor allows for reduced formation of complex during the reading period, while allowing for a robust signal, providing at least a five-fold, usually at least a 10-fold of ratio of signal to background during the period of the reading. One or more readings will be taken within 150 min, more usually within 120 min, preferably within about 60 min, and usually after about 10 min, more usually after about 15 min.

While various intracellular events are of interest, of primary interest are translocations. In this assay, little, if any, formation of the active enzyme occurs without there being translocation of the fusion protein. The barrier to formation will be a physical barrier, where the unfused fragment is retained in a cellular compartment. Only when the fused protein is translocated to the compartment will active enzyme complex formation occur to a significant degree. Of particular interest are surface membrane proteins that upon activation are transported to a cellular compartment. Cellular compartments may include mitochondria, chloroplasts, the cell nucleus, the Golgi apparatus, vesicles, and microtubules, as well as the cytosol itself.

Any eukaryotic cell may be employed, for the most part cell lines being employed. The cell lines will usually be mammalian, but for some purposes unicellular organisms or cells from non-vertebrates can be used. Mammalian cell lines include CHO, HeLa, MMTV, and the like. The cells are genetically modified transiently or permanently, usually permanently. Various vectors that are commercially available can be used successfully to introduce the two expression constructs into the eukaryotic cell. For an extensive description of cell lines, vectors, methods of genetic modification, and expression constructs, see published US application serial no. 2003/0092070, Zhao, et al., May 15, 2003, paragraphs 00046-00066, which are specifically incorporated herein by reference.

There may be some selection as to the relative strength of the promoters for the two fragments. Desirably, the unfused fragment promoter should not be too much stronger than the fused fragment promoter, generally providing an expression ratio of less than about 20, usually less than about 10, between expression of the two proteins. Determining the expression level of a promoter is well within the skill of the art, conveniently using labeled antibodies to the two proteins in conventional assays.

The reagent solution is a high ionic strength solution to allow for interaction between the enzyme substrate and the enzyme formed intracellularly through the cell membrane. In this way, any active enzyme complex that is formed as a result of cell stimulation and translocation can be detected by the signal resulting from the product. The assay depends upon using high salt concentration, particularly sodium chloride, in conjunction with minor amounts of other salts. Generally, the molarity of the high ionic strength reagent solution will be in excess of 100 mM and not more than about 350 mM, usually in the range of about 150 to 250 mM. Sodium chloride will be at least 50% of the total salts, more usually at least about 60%, and generally not more than about 90%, generally ranging from about 100 to 300 mM. The auxiliary salts will serve a variety of purposes besides moderately enhancing the ionic strength. These salts will generally range in an amount of from about 1 to 5% each of the total salts. At these salt concentrations the enzyme is active and the enzyme product is readily detectable. These salts include potassium salts, particularly phosphate salts, which at the pH of the reagent solution and the assay solution will be a mixture of mono- and dibasic phosphate, and magnesium salts, particularly magnesium acetate, which aids in the enzyme activity. The phosphate salts serve also as buffering agents and will be present in a total of about 5-20 mM. The amount is not critical, so long as the pH is maintained under the conditions of the assay. The pH will generally be in the range of about 6.5 to 8, more usually 6.7 to 7.5, desirably at about 6.9.

In addition to the salts, an organic detergent is employed. Of particular interest are neutral, e.g. zwitterions, detergents. Various detergents may be employed, such as CHAPS (3-[(3-cholamidopropyl)dimethylammonio]-1-propanesulfonate), CHAPS being preferred. The concentration of the detergent will generally be from about 1 to 5, more usually 1 to 3, and particularly 2, percent of the reagent solution.

Also present will be a luminescent reagent comprising a β-galactosidase substrate and optionally a signal enhancer. The luminescent reagent will be in large excess in relation to the maximum amount of β-galactosidase that is likely to be formed. Conveniently, a luminescent substrate is used, available as Galacton Star from ABI in conjunction with the Emerald II enhancer. Any equivalent luminescent substrate composition may be employed. The substrate will be present in about 1 to 10 weight percent, while the enhancer will be present in about 10 to 30 weight percent of the reagent solution. These amounts will vary depending upon the particular substrate composition employed.

The reagent solution may be prepared as a 5-20× concentrate or higher for sale or the solids may be provided as powders and dissolved in water at the appropriate proportions.

The cells for use in the assay will be grown in accordance with the nature of the cells. For the most part, cells will be grown in wells in microtiter plates, the number of wells generally ranging from about 96 to 1536, generally being from 96 to 384 wells. The bottom will generally be clear, so that readings may be taken from the bottom of the wells. The number of cells plated in a well will generally range from about 10² to 10⁴ cells. The volume of the medium will be of small volume, usually in the range of about 50 to 200 μl, so that the medium will be the lesser portion of the total volume when the reagent solution is added. The cells are then allowed to adhere overnight using conventional conditions of 37° C./5% CO₂.

To the adherent cells is added a compound of interest, usually an organic compound that is being screened for its ability to stimulate the cell and initiate translocation of a protein or alternatively inhibit a compound that stimulates the cell. The concentration of the compound of interest will vary widely depending on the nature of the target, the activity range of interest, solubility in an aqueous medium, etc. Generally, the volume of the agent added will be in the range of about 5 to 50 μl. The mixture will be incubated for a reasonable time, again based on the particular study being performed. Incubations will generally be at least about 0.5 h and usually not exceed 24 h, generally be in the range of about 1-6 h.

After sufficient time for the stimulation of the cells to take effect and the target protein to be translocated, a large volume of the reagent solution is added generally being at least about 2×, more usually at least about 3×, and not more than about 10×, usually not more than about 5×, the cell medium. Generally, the reagent solution volume will be in the range of about 200 to 600 μl, more usually in the range of about 300 to 500 μl. It is found that the formed enzyme complex is retained, the potential for new complex to form as a result of the permeabilizing of the cells is inhibited and the background from other than complex formed from the translocation is minimal. In this way a robust response to the activity of the stimulation is achieved. No further additions are required. After sufficient time for the enzyme reaction to have stabilized, generally 5 to 60 min, one or more readings may be taken. A conventional commercially available luminescent plate reader can be used effectively.

Standards will usually be used, whereby the signal is related to the concentration of a known stimulator performed under the same conditions as the candidate compound. A graph can be prepared that shows the change in signal with the change in concentration of the standard compound.

Kits can be provided that include the reagent solution or powders of the components in appropriate proportions, where normally not more than about a 10% excess of the ingredients may be provided. The cells may be included in the kit or provided separately, where the recipient is able to continuously expand the cell population while exhausting the reagents. Also, a standard can be provided, as a graph or as the compound to be used for comparison, as well as directions for performing the assay. The directions may be written (hard copy) or electronic, e.g. a CD (soft copy).

The following examples are offered by way of illustration and not by way of limitation.

### EXPERIMENTAL

The following are the composition of the reagent solution and the protocol used with glucocorticoid receptor fused to ED.

Reagent Solution Buffer Formulation for GR Translocation Assay:

5 mM Potassium phosphate (dibasic)
5 mM Potassium phosphate (monobasic)
150 mM Sodium Chloride
10 mM Magnesium Acetate
2% CHAPS
pH6.9
   Reagent Buffer/Substrate Formulation/Mixture Used in the Assay:
1 part Galacton Star (ABI)
5 parts Emerald II enhancer (ABI)
19 parts Lysis buffer with 2% CHAPS (DX)
   Assay Protocol (96 Well Assay Format)
1. 10,000 CHO-EA+GR-PL cells/well in a volume of 100 μL are plated in a Costar 96 well white, clear bottom plate and allowed to adhere overnight at 37° C./5% CO₂ in an incubator.
2. Next day, 20 μLs of dexamethasone {Sigma, cat #D8893} (made up at a reagent concentration of 5% Ethanol diluted in F12 media, to yield a final concentration of 1% ethanol in the reaction) at the desired concentration is added to the cells.
3. The reaction is incubated for 3-4 hours in a 37° C./5% CO₂ incubator.
4. To the reaction, equal aliquots from 50 mL of reagent solution are added to the wells.
5. The plate is read on a standard multiwell plate reader that can measure luminescent activity. (e.g., Wallac Victor II or Packard Lumicount) after 30 and 60 minutes.

The following is a description of the preparation of the CHO-EA+GR-PL cells, with a description of the preparation of the constructs, introduction into the vectors, and introduction of the vectors into the CHO cells. Also described is the screening of the cells for the appropriate activity.

EA-NLS/NRS Construct

The initial ω or EA fragment of beta-galactosidase was generated by using a Stanford generated EA-NLS/NRS construct as the pcr template (Wehrman, T. S., Casipit, C. L., Gewertz, N. M., Blau, H. M., Nature methods, 2, 521-527, 2005). Kpn I and Not I restriction sites were engineered on the 5′ and 3′ ends respectively by the pcr reaction. This EA fragment was subcloned into the Invitrogen vector, pcDNA3.1 hygro (cat #V870-20). This plasmid construct was used to transfect CHO-K1 cells and to eventually select a hygromycin resistant clonal cell that expresses EA localized in the nucleus.

Clonal selection was performed by transfecting the cells with FuGene6 reagent (Roche), letting the transfection go for 48 hours and then subjecting the cells to increasing amounts of hygromycin (100, 250, 400 μg/mL) to place selective pressure on the cells that had taken up the EA-NLS/NRS plasmid. After two weeks in selection, the EA-NLS/NRS cDNA should have integrated into the CHO-K1 genome via a homologous recombination event to yield a hygromycin resistant pool population of cells that express EA-NLS/NRS. Serial dilutions of the selected pool cells were plated in a 96 well plate and putative clones, which were derived from a single cell, were identified and then characterized. Characterization of a clone involved generation of a lysate from the clonal cells and the addition of exogenous ED reagent (at 1, 10, 100 nM concentration) and chemiluminescent substrate to measure total substrate turnover. From these results a hierarchy of EA expressing clones were selected and further characterized for the ability to interact with other ED-tagged genes (ED is commercially referred to as PL or ProLabel.).

A second ω or EA fragment of beta-galactosidase was generated using a DiscoveRx pCMV-EA construct as the pcr template and generated by Panomics, Inc. Kpn I and Not I restriction sites were engineered on the 5′ and 3′ ends respectively by the pcr reaction. This EA fragment was subcloned into the Invitrogen vector, pcDNA3.1 hygro (cat #V870-20). To the 3′ end of EA, a triple SV40 nuclear localization sequence {NLS} (generated by using the Invitrogen plasmid pCMV/myc/nuc, cat #V821-20 as a pcr template) was subcloned into the Not I and Xho I sites of the EA plasmid. Finally, to this construct, a nuclear retention signal {NRS} from SC35 was generated using an Open Biosystems cDNA clone (IMAGE clone # 3452024) as a template for pcr and then subcloned into the Xho I and Xba I sites of the EA-NLS plasmid construct. A final variation on this construct was to add a c-myc sequence ((DLKVRKAA) SEQ ID NO: 1 which has been demonstrated to help localize proteins to the nucleus (Saphire, A. C. S., Bark, S. J., Gerace, L., JBC, 273, 29764-29769, 1998)), which sequence was subcloned into the EA-NLS/NRS construct. This plasmid was used to transfect CHO-K1 cells that were then subjected to antibiotic treatment and eventual single cell clonal selection to generate the DX EA-Nuc parental cell line performed as described in the above paragraph.

GR-PL-myc Plasmid Construct

The glucocorticoid receptor (GR) was subcloned by using a pCMV-GR-PL construct generated by Panomics, Inc. as pcr template, adding Bgl 2 and Kpn I sites at the 5′ and 3′ ends respectively. The GR sequence was based on and confirmed to match the RefSeq NM_{—}000176 for the human GR. The GR cDNA was subcloned into the multiple cloning site (Bgl 2 and Kpn I sites) of a pCMV based plasmid which already contained the α or ED/ProLabel fragment of β-galactosidase along with a myc epitope tag (EQKLISEEDL) SEQ ID NO: 2 at the 3′ end of ProLabel. This plasmid was transiently transfected into a stable CHO-K1+EA-NLS/NRS expressing cell line. Clonal selection for cells expressing both EA-NLS/NRS and GR-PL was performed as described above except the cells were subjected to double antibiotic selection (hygromycin and Geneticin/G418). The clone was confirmed by EFC (Enzyme Fragment Complementation) measurement, Eastern™, Western, immunofluorescence imaging using α-myc, α-beta galactosidase and α-GR antibodies, as well as by functional pharmacology testing for a response by the cells to dexamethasone treatment.

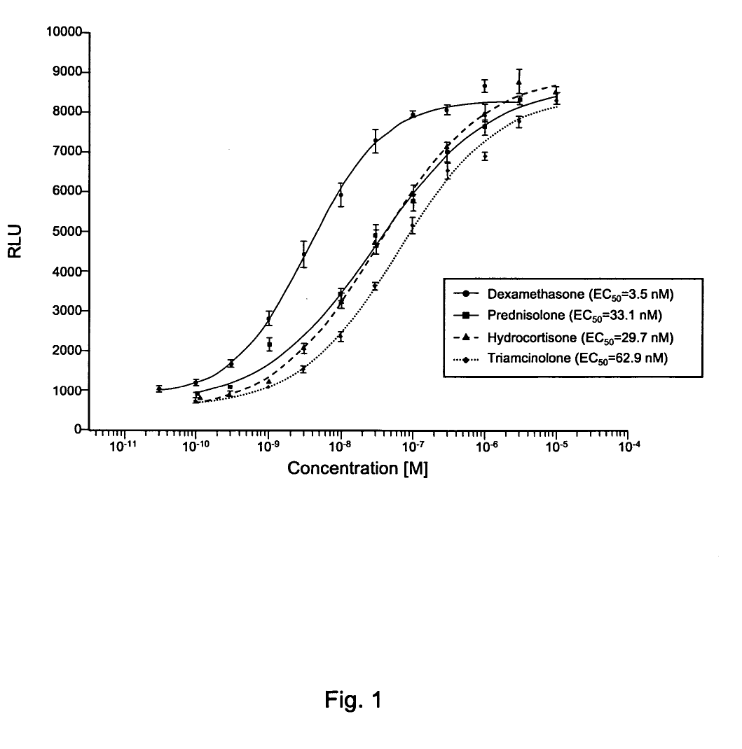

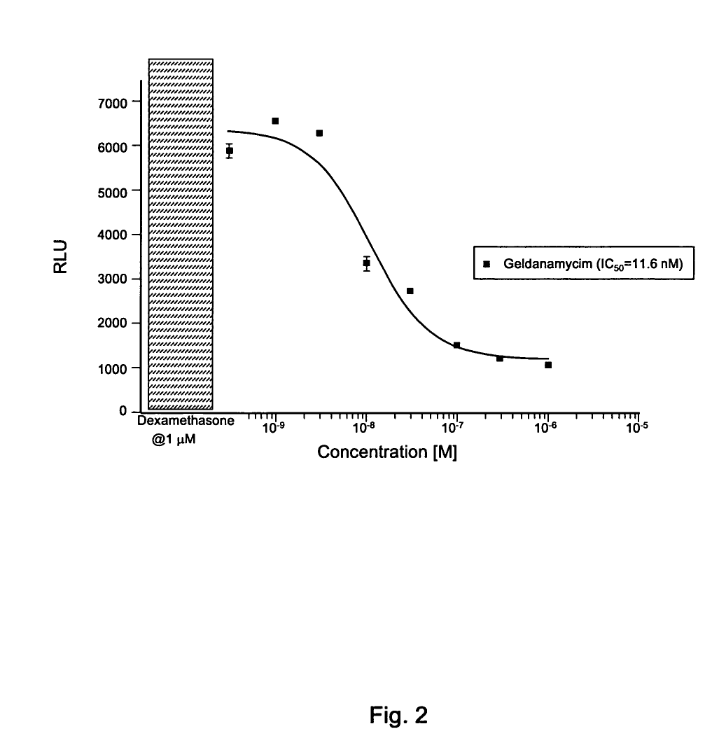

10,000 cells/well were plated in a white Costar® 96 well plate and allowed to adhere overnight. After the cells adhered overnight, GR agonists, dexamethasone, hydrocortisone, prednisolone and triamcinolone were added to the PathHunter cells for 3 hours and the EFC activity measured. See FIG. 1. The cells were incubated with the dexamethasone or other agonist for 3 hours at 37° C./5% CO₂. To the reaction, 50 μL of cell lysis/substrate solution was added and the plate was read in a Wallace Victor 2 plate reader after 60 minutes. The EC50 of dexamethasone, prednisolone, hydrocortisone and triamcinolone was 3.5, 33.1, 29.7 and 62.9 nM respectively. The signal to noise ratio of the compounds was ˜8.5. Antagonist studies were also performed using titrating amounts of geldenamycin (1-1000 nM) pre-incubated with the PathHunter cells for 1 hour prior to being treated with an agonist (at the EC80 concentration) for 3 hours and the EFC activity was measured. An IC₅₀ of 11.6 nM for geldenamycin was obtained with a dexamethasone treatment as shown in FIG. 2. Using 1 μM of the different agonists, prednisolone and hydrocortisone, following the above procedure, IC₅₀s were obtained of 22.3 and 23.8 nM respectively.

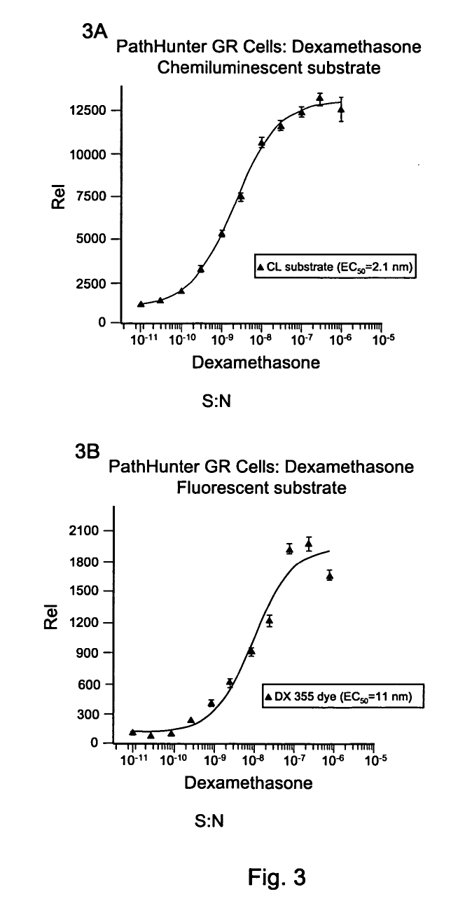

In the next study, using the procedure described above for dexamethasone treatment, chemiluminescent and fluorescent detection of the PathHunter GR Cell assay were compared. The PathHunter GR Cell assay is simple to run and amenable to both CL and FL detection. In the chemiluminescent mode, dexamethasone had an EC50 of 2.1 nM and a signal to noise ratio of 12.3. In the fluorescent mode, dexamethasone had an EC50 of 11 nM and a signal to noise ratio of 16.2. The PathHunter GR Cell assay can be measured in both CL and FL modes with very robust signal production and no compromise to the assay performance. The results are shown in FIGS. 3A and 3B.

It is evident from the above results that the subject invention provides for a sensitive, accurate method for determining cellular events, particularly translocation where the two fragments of β-galactosidase are prevented from forming an active enzyme complex by means of a cellular barrier. The method finds particular application in cells for translocation to the nucleus. The protocol is simple, easily and accurately performed and the reagents are robust and provide for a robust signal at low concentrations of candidate compounds.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for detecting translocation of a protein in response to a stimulus employing genetically modified cells having: a first expression construct encoding for a first β-galactosidase fragment fused to a protein of interest, where said protein of interest translocates upon said stimulus to a cellular compartment, and a second expression construct encoding a second β-galactosidase fragment fused to a signal sequence directing said second β-galactosidase to said cellular compartment, except when said cellular compartment is the cytosol, wherein said first and second fragments independently complex to form an active enzyme and said compartment provides a physical barrier to said first and second β-galactosidase fragments forming an active enzyme when said first β-galactosidase fragment is not present in said cellular compartment, said method comprising:
contacting said cells in a medium of small volume with a candidate stimulus for sufficient time for any translocation to occur;
adding a high ionic strength reagent comprising an enzyme substrate composition resulting in a luminescent product producing a luminescent signal within a time period providing at least about a 5:1 signal:noise ratio;
wherein a positive luminescent signal indicates that said candidate stimulus stimulated said cells resulting in said translocation of said protein of interest.

2. A method according to claim 1, wherein said protein of interest is a surface membrane protein that translocates to the nucleus and said second fragment is located in said nucleus.

3. A method according to claim 1, wherein said reagent solution results in a dilution of said small volume and high strength reagent is not more than about 1:2.

4. A method according to claim 1, wherein said first fragment is a small fragment of β-galactosidase.

5. A method according to claim 1, wherein the reagent is an aqueous solution comprising from about 100 to 300 mM NaCl.

6. A method according to claim 5, wherein the molarity of said reagent solution is in the range of about 100 to 350.

7. A method according to claim 6 comprising from 1 to 3% of a zwitterionic detergent.

8. A method for detecting translocation to the nucleus of a surface membrane protein receptor in response to a ligand binding to said receptor employing genetically modified cells having: a first expression construct encoding for a first β-galactosidase small fragment fused to said receptor, and a second expression construct encoding a second large β-galactosidase fragment fused to a signal sequence directing said second β-galactosidase to said nucleus, wherein said first and second fragments independently complex to form an active enzyme and said nucleus provides a physical barrier to said first and second β-galactosidase fragments forming an active enzyme when said first β-galactosidase fragment is not present in said nucleus, said method comprising:
contacting said cells in a medium of small volume with a candidate stimulus for sufficient time for any translocation to occur;
adding a high ionic strength reagent solution comprising an enzyme substrate composition resulting in a luminescent product producing a luminescent signal within a time period providing at least about a 5:1 signal:noise ratio;
wherein a positive luminescent signal indicates that said candidate stimulus stimulated said cell resulting in said translocation of said receptor.

9. A method according to claim 8, wherein said reagent solution results in a dilution of not more than about 1:2 of said medium and reagent solution.

10. A method according to claim 8, wherein said first fragment is a small fragment of β-galactosidase.

11. A method according to claim 8, wherein the reagent solution comprises from about 100 to 300 mM NaCl.

12. A method according to claim 11, wherein the molarity of said reagent solution is in the range of about 100 to 350.

13. A method according to claim 12 comprising from 1 to 3% of a zwitterionic detergent.

14. A method according to claim 13, wherein said detergent is CHAPS.

15. A method according to claim 14, wherein said receptor is the glucocorticoid receptor.

16. A reagent solution, for use in a method according to claim 1, having a high ionic strength comprising from about 100 to 300 mM NaCl, a phosphate buffer, a magnesium salt and a zwitterionic detergent, in combination with directions for performing said method.

17. A reagent solution according to claim 16, wherein said zwitterionic detergent is CHAPS in an amount from about 1 to 3%.

18. A reagent solution according to claim 16, wherein said directions are in electronic readable form.


SEQUENCE LISTING (2 sequences)

SEQ ID NO: 1 - protein, 8 aa, Artificial Sequence
  c-myc subsequence
DLKVRKAA

SEQ ID NO: 2 - protein, 10 aa, artificial sequence
  c-myc epitope tag
EQKLISEEDL